(19) United States
(12) Patent Application Publication
(10) Pub. No.: US 2026/0041676 A1
(43) Pub. Date: Feb. 12, 2026

(54) Combination Therapy

(71) Applicants: Medivation Prostate Therapeutics LLC, New York, NY (US); Astellas Pharma Inc., Tokyo (JP)

(72) Inventors: Jacqueline Gibbons, San Franciso, CA (US); Joyce Mordenti, San Francisco, CA (US); Michiel De Vries, Leiden (NL); Walter Krauwinkel, Leiden (NL); Taoufik Ouatas, Bizerte (TN)

(21) Appl. No.: 19/070,302

(22) Filed: Mar. 4, 2025

Related U.S. Application Data
(63) Continuation of application No. 18/808,190, filed on Aug. 19, 2024, now abandoned.
Application No. 18/808,190 is a continuation of application No. 18/433,659, filed on Feb. 6, 2024, now abandoned.
Application No. 18/433,659 is a continuation of application No. 18/209,248, filed on Jun. 13, 2023, now abandoned.
Application No. 18/209,248 is a continuation of application No. 17/979,063, filed on Nov. 2, 2022, now abandoned.
Application No. 17/979,063 is a continuation of application No. 17/729,194, filed on Apr. 26, 2022, now abandoned.
Application No. 17/729,194 is a continuation of application No. 16/833,819, filed on Mar. 30, 2020, now abandoned.
Application No. 16/833,819 is a continuation of application No. 16/543,753, filed on Aug. 19, 2019, now abandoned.
Application No. 16/543,753 is a continuation of application No. 16/231,632, filed on Dec. 24, 2018, now abandoned.
Application No. 16/231,632 is a continuation of application No. 15/751,610, now abandoned.
(60) Provisional application No. 62/204,287, filed on Aug. 12, 2015.

(51) Int. Cl. A61K 31/4439; A61K 45/06

(52) U.S. Cl. CPC A61K 31/4439; A61K 45/06

(57) ABSTRACT

This disclosure provides a dosage regimen for co-administration of 4-[7-[6-cyano-5-(trifluoromethyl)pyridin-3-yl]-8-oxo-6-sulfanylidene-5,7-diazaspiro[3.4]octan-5-yl]-2-fluoro-N-methylbenzamide and a strong CYP3A4 inducer.

This application is a continuation of Ser. No. 18/808,190 filed Aug. 19, 2024, which is a continuation of Ser. No. 18/433,659, filed Feb. 6, 2024, which is a continuation of Ser. No. 17/979,063, filed Nov. 2, 2022, which is a continuation of Ser. No. 16/543,753, filed Aug. 19, 2019, which is a continuation of Ser. No. 16/231,632, filed Dec. 24, 2018, which is a continuation of Ser. No. 15/751,610. Ser. No. 15/751,610 is a U.S. national phase application of PCT/US16/46470, filed on Aug. 11, 2016. PCT/US16/46470 claims priority to and incorporates by reference U.S. provisional application Ser. No. 62/204,287, filed on Aug. 12, 2015.

### TECHNICAL FIELD

This disclosure relates generally to cancer treatment.

### DETAILED DESCRIPTION

4-[7-[6-cyano-5-(trifluoromethyl)pyridin-3-yl]-8-oxo-6-sulfanylidene-5,7-diazaspiro[3.4]octan-5-yl]-2-fluoro-N-methylbenzamide is an androgen receptor inhibitor and can be used to treat cancers such as prostate cancers, breast cancers, and ovarian cancers. If 4-[7-[6-cyano-5-(trifluoromethyl)pyridin-3-yl]-8-oxo-6-sulfanylidene-5,7-diazaspiro[3.4]octan-5-yl]-2-fluoro-N-methylbenzamide is co-administered with a strong CYP3A4 inducer (e.g., carbamazepine, phenobarbital, phenytoin, rifabutin, rifampin, rifapentine), the daily dose of 4-[7-[6-cyano-5-(trifluoromethyl)pyridin-3-yl]-8-oxo-6-sulfanylidene-5,7-diazaspiro[3.4]octan-5-yl]-2-fluoro-N-methylbenzamide may be increased from, e.g., 160 mg/day to 200-300 mg/day (e.g., 200, 205, 210, 215, 220, 225, 230, 235, 240, 245, 250, 255, 260, 265, 270, 275, 280, 285, 290, 295, 300 mg/day).

“Co-administration” of 4-[7-[6-cyano-5-(trifluoromethyl)pyridin-3-yl]-8-oxo-6-sulfanylidene-5,7-diazaspiro[3.4]octan-5-yl]-2-fluoro-N-methylbenzamide and a strong CYP3A4 inducer means administration in any manner in which the pharmacological effects of 4-[7-[6-cyano-5-(trifluoromethyl)pyridin-3-yl]-8-oxo-6-sulfanylidene-5,7-diazaspiro[3.4]octan-5-yl]-2-fluoro-N-methylbenzamide and the strong CYP3A4 inducer overlap in the patient at the same time. Co-administration does not require that both agents be administered in a single pharmaceutical composition, in the same dosage form, by the same route of administration, or for the same length of time.

4-[7-[6-cyano-5-(trifluoromethyl)pyridin-3-yl]-8-oxo-6-sulfanylidene-5,7-diazaspiro[3.4]octan-5-yl]-2-fluoro-N-methylbenzamide is typically formulated for oral administration, for example, in solution in caprylocaproyl polyoxylglycerides.

Patients who can be treated with the disclosed co-administration regimes include patients with prostate cancer (including metastatic prostate cancer, castration-resistant prostate cancer, hormone-sensitive prostate cancer, metastatic castration-resistant prostate cancer, metastatic hormone-sensitive prostate cancer), breast cancer (including triple-negative breast cancer), and ovarian cancer. Prostate cancer patients who can be treated using the disclosed co-administration regimes include patients with metastatic castration-resistant prostate cancer (CRPC) who had previously received chemotherapy (e.g., docetaxel) as well as patients with CRPC who are chemotherapy-naïve.

1. A method of treating cancer in a patient to whom a CYP3A4 inducer is administered, comprising administering to the patient a therapeutically effective dose of 4-[7-[6-cyano-5-(trifluoromethyl)pyridin-3-yl]-8-oxo-6-sulfanylidene-5,7-diazaspiro[3.4]octan-5-yl]-2-fluoro-N-methylbenzamide and a CYP3A4 inducer, wherein the therapeutically effective dose of 4-[7-[6-cyano-5-(trifluoromethyl)pyridin-3-yl]-8-oxo-6-sulfanylidene-5,7-diazaspiro[3.4]octan-5-yl]-2-fluoro-N-methylbenzamide is 200-300 mg per day.

2. The method of claim 1, wherein the cancer is selected from the group consisting of prostate cancer, breast cancer, and ovarian cancer.

3. The method of claim 1, wherein the therapeutically effective dose of 4-[7-[6-cyano-5-(trifluoromethyl)pyridin-3-yl]-8-oxo-6-sulfanylidene-5,7-diazaspiro[3.4]octan-5-yl]-2-fluoro-N-methylbenzamide is 240 mg per day.

4. The method of claim 2, wherein the cancer is prostate cancer, and the prostate cancer is castration-resistant prostate cancer.
